(19) United States
(12) Patent Application Publication
(10) Pub. No.: US 2021/0322707 A1
(43) Pub. Date: Oct. 21, 2021

(54) VENTILATOR SPLITTER MODULE AND SHARING SYSTEM CONFIGURED TO CONNECT MULTIPLE PATIENTS TO A SINGLE VENTILATOR WITH INDEPENDENT VENTILATION PARAMETER CONTROL

(71) Applicants: BLOOM ENERGY CORPORATION, San Jose, CA (US); THE BOARD OF TRUSTEES OF THE LELAND STANFORD JUNIOR UNIVERSITY, Stanford, CA (US)

(72) Inventors: Swaminathan VENKATARAMAN, Cupertino, CA (US); Tyler DAWSON, Sunnyvale, CA (US); Shannon BELL, San Jose, CA (US); Evan LY, San Jose, CA (US); Jordan CEDARLEAF-PAVY, Sunnyvale, CA (US); Victor SILVA, San Jose, CA (US); Jayakumar KRISHNADASS, Sunnyvale, CA (US); Prasad PMSVVSV, Sunnyvale, CA (US); Ali ZARGARI, Santa Clara, CA (US); David EDMONSTON, Soquel, CA (US); Michael PAULSEN, Palo Alto, CA (US); Joseph WOO, Palo Alto, CA (US); Annabel IMBRIE-MOORE, Standford, CA (US); Jonathan GARDNER, Sun Valley, ID (US); Jerome MACK, San Carlos, CA (US)

(21) Appl. No.: 17/229,245

(22) Filed: Apr. 13, 2021

Related U.S. Application Data
(60) Provisional application No. 63/010,580, filed on Apr. 15, 2020.

(51) Int. Cl. A61M 16/08; A61M 16/20; A61M 16/10; A61M 16/00

(52) U.S. Cl. CPC A61M 16/0875; A61M 16/201; A61M 16/0051; A61M 16/107; A61M 16/1065

(57) ABSTRACT

A splitter module is configured to connect a single medical ventilator to multiple intubated patients. The splitter module is configured to independently control at least one ventilation parameter for each of the patients, such that modifying a ventilation parameter of one of the patients does not significantly affect the ventilation parameters of the other patients.

### FIELD

Aspects of the present disclosure relate to medical devices, such as ventilator splitter devices and methods of using the same.

### BACKGROUND

The novel coronavirus SARS-CoV-2 often results in severe respiratory disease with acute respiratory distress syndrome (ARDS). ARDS often results in impaired gas exchange and decreased lung compliance. Mechanical ventilation is the mainstay of treatment for ARDS. There is a global shortage of mechanical ventilators to treat patients with the severe respiratory disease.

### SUMMARY

According to various embodiments, a splitter module is configured to connect a single medical ventilator to multiple intubated patients, wherein the splitter module is configured to independently control at least one ventilation parameter for each of the patients, such that modifying a ventilation parameter of one of the patients does not significantly affect the ventilation parameters of the other patients.

According to various embodiments, a ventilator sharing and monitoring system (VSMS) comprises a medical ventilator; a splitter module fluidly connected to the medical ventilator, and comprising a inhalation manifold configured to divide an inspiration stream received from the medical ventilator into separate inhalation streams; plural inhalation lines fluidly connected to the splitter module and configured to provide the inhalation streams to respective intubated patients; plural exhalation lines configured to receive respective exhalation streams exhaled from the patients; and an exhalation manifold fluidly connected to the plural exhalation lines and to the medical ventilator, and configured to combine the exhalation streams into an expiration stream, and provide the expiration stream to the medical ventilator.

According to various embodiments, provided is a medical ventilation method comprising: connecting a single medical respirator to intubated patients through a splitter module; and adjusting a ventilation parameter of one of the patients using the splitter module, without significantly affecting ventilation parameters of the remaining patients.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1A is a schematic view showing air flow through a ventilator sharing and monitoring system (VSMS) 300, FIG. 1B is a schematic view showing components of the VSMS 300 of FIG. 1A, FIG. 1C is a schematic cut-away top view of the splitter module 100 of the VSMS 300 of FIGS. 1A and 1B, and FIG. 1D is a schematic view showing power architecture of the VSMS 300 of FIG. 1B, according to various embodiments of the present disclosure.

FIG. 2 shows VSMS data flow from user input and data acquisition for analysis and display.

FIG. 3 is a screen shot showing ventilation parameters that may be displayed by the VSMS, according to various embodiments of the present disclosure.

FIGS. 4A-4D are plots of individual patient pressure and tidal volume exhale (in units of centimeters of water column at 4° C. (“cm H₂O”) and mL, respectively) versus time in seconds that may be generated and displayed by the VSMS during in-line monitoring of patients.

FIGS. 5A-5C are photographs showing outer surfaces of a splitter module 500, according to various embodiments of the present disclosure, that exemplifies a splitter module 100 of FIGS. 1A-1D.

FIG. 6A is a photograph showing the exemplary splitter module 500 with its cover removed, and 6B is a photograph showing the exemplary splitter module 500 with its cover and mid plane removed, according to various embodiments of the present disclosure.

FIG. 7A is a photograph of a patient ventilation circuit 210, FIG. 7B is a photograph of a one-way valve, and FIG. 7C is a photograph of a pressure line, according to various embodiments of the present disclosure.

FIG. 8 is a photograph of an exhalation manifold 250, according to various embodiments of the present disclosure.

FIGS. 9A, 9B, and 9C are side and perspective views of a mounted splitter module, according to various embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

Prior art ventilators are set up to only be able to manipulate the pressure and volume for an individual patient. Prior art ventilators support the use of simple splitters to assist supporting multiple patients but provide no independent control for each patient.

According to one embodiment of the present disclosure, a medical ventilator splitter device allows clinicians to control the ventilator parameters (such as peak pressure and positive end-expiratory pressure) to different patients based on their needs without affecting other patients on the splitter device. Thus, the splitter device gives the ability to support many patients on a single ventilator and also control each patient's breathing needs.

The ventilator splitter device of the embodiments of the present disclosure gives the operator control of the individual pressure and volume requirements to more than one patient that is using a single ventilator (e.g., plural intubated patients which are connected to the medical ventilator through respective breathing tubes and the splitter device). This splitter device and method can be used in cases where ventilators are in short supply. The splitter device can be used with all ventilator models regardless of whether they have open or closed loop control.

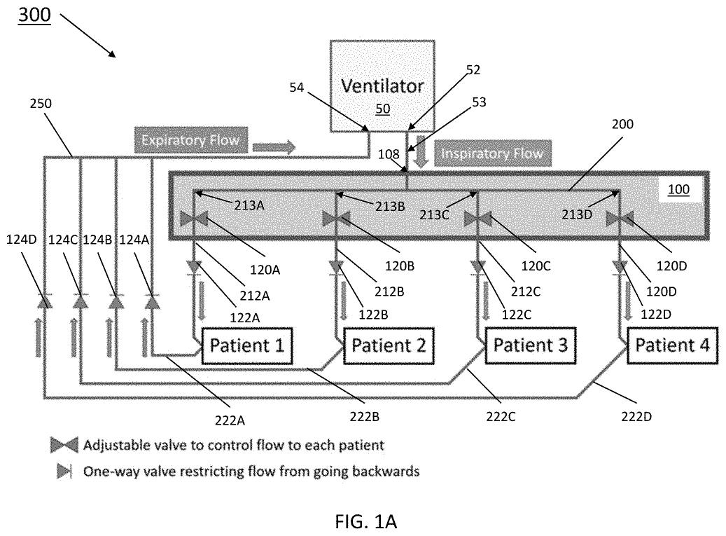

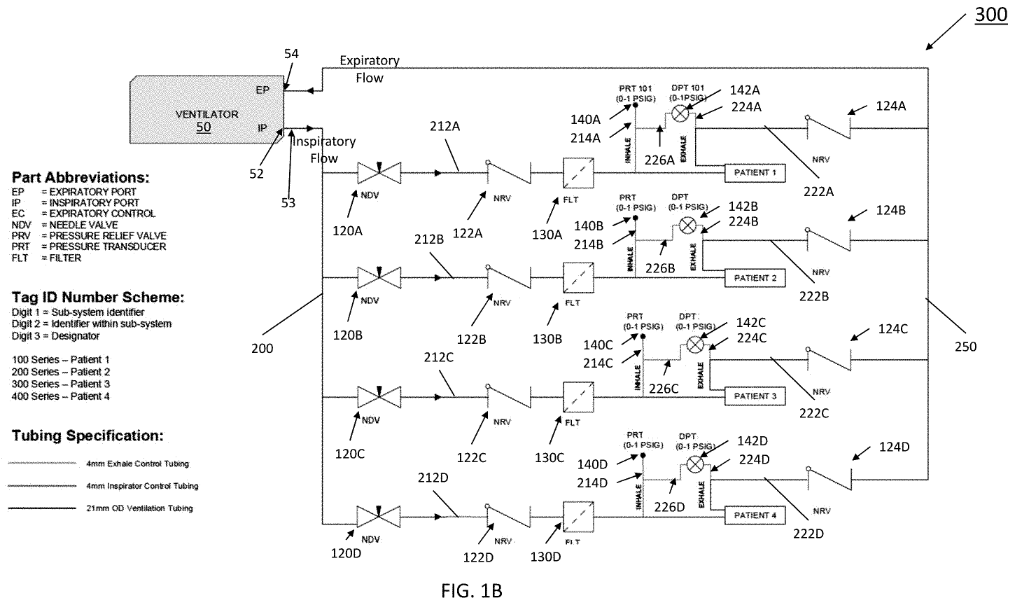

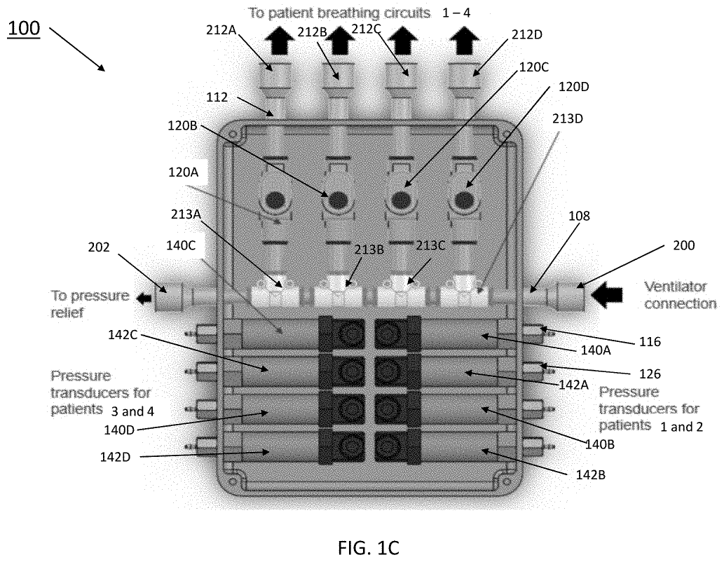

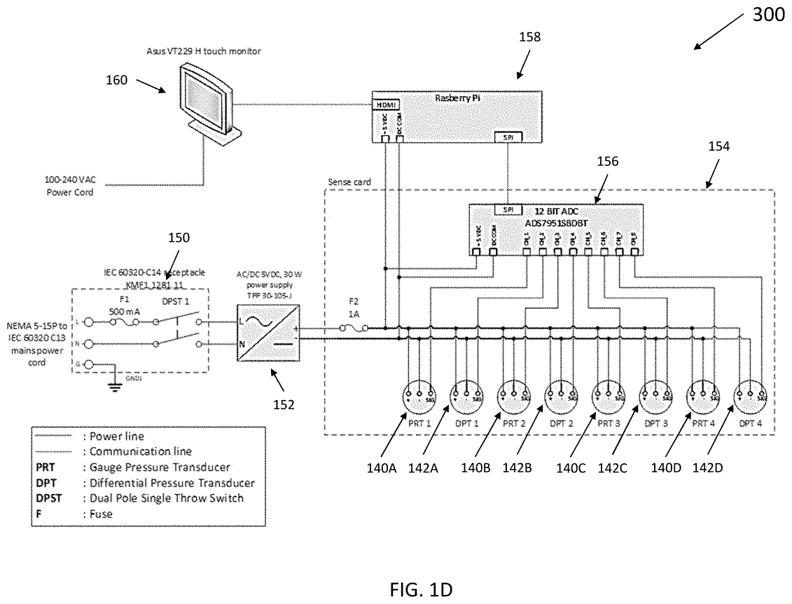

FIG. 1A is a schematic view showing air flow through a ventilator sharing and monitoring system (VSMS) 300, according to various embodiments of the present disclosure. FIG. 1B is a schematic view showing components of the VSMS 300 of FIG. 1A. FIG. 1C is a schematic cut-away top view of the splitter module (i.e., splitter device) 100 of the VSMS 300 of FIGS. 1A and 1B, according to various embodiments of the present disclosure.

Referring to FIGS. 1A, 1B and 1C, the VSMS 300 may include a ventilator 50 and a splitter module 100. The splitter module 100 may include an inhalation manifold 200 that is connected to an inhalation port 52 of the ventilator 50. The inhalation manifold 200 may be fluidly connected to one or more patients, such as patients 1-4, by respective inhalation lines 212A-212B. In particular, the inhalation manifold 200 may be configured to divide an inspiration stream received from the ventilator 50 into inhalation streams that are respectively provided to the patients 1-4.

The patients 1-4 may be connected to an exhalation manifold 250 by respective exhalation lines 222A-222B. The exhalation manifold 250 may be fluidly connected to an exhalation port 54 of the ventilator 50. In particular, the exhalation manifold 250 may combine exhalation streams exhaled from the patients 1-4 into an expiration stream, and provide the expiration stream to the ventilator 50.

Accordingly, the VSMS 300 may be configured to ventilate multiple patients using a single ventilator 50, by dividing an inspiratory air stream output from the ventilator 50 into separate patient air streams, and providing each patient air stream to a respective patient. While four patients are shown, the present disclosure is not limited to any particular number of patients. For example, the VSMS 300 may be used to ventilate a single patient, two, three, four, or more than four patients, depending upon the ventilation capacity of the ventilator 50.

Pressure control valves 120A-120D may be fluidly connected to the respective inhalation lines 212A-212D which are fluidly connected to the inhalation manifold 200 at respective junctions 213A-213D, which may be T-shaped junctions and/or elbow junctions. One-way inhalation valves 122A-122B and filters 130A-130D may also be fluidly connected to respective inhalation lines 212A-212D. The inhalation manifold 200 may also have an optional pressure relief outlet 202 controlled by a pressure relief valve (not shown in FIG. 1C). One-way exhalation valves 124A-124D may be fluidly connected to the respective exhalation lines 222A-222D which are fluidly connected to the exhalation manifold 250.

The pressure control valves 120A-120D may be needle valves configured to control the pressure and/or flow rate of inhalation flow streams through each inhalation line 212A-212D. The pressure control valves 120A-120D can be dialed by an operator to a corresponding patient's specific air pressure needs. The filters 130A-130D may be, for example HEPA filters, configured to filter air provided to the patients 1-4. The inhalation valves 122A-122D may be non-return valves configured to prevent air exhaled from the patients 1-4 from flowing back into the inhalation lines 212A-212D. The exhalation valves 124A-124D may be non-return valves configured to prevent exhaled air from returning to the patients 1-4 from the exhalation manifold 250.

The VSMS 300 may also include gauge pressure transducers 140A-140D and differential pressure transducers 142A-142D. The gauge pressure transducers 140A-140D may be respectively fluidly connected to the inhalation lines 212A-212D by inhalation pressure lines 214A-214D. The differential pressure transducers 142A-142D may be respectively fluidly connected to the exhalation lines 222A-222D by exhalation pressure lines 224A-224D. The differential pressure transducers 142A-142D may be respectively fluidly connected to the inhalation pressure lines 214A-214D by differential lines 226A-226D.

The gauge pressure transducers 140A-140D may be configured to detect inhalation parameters, such as an inhalation pressure within the respective inhalation lines 212A-212D. In other words, the gauge pressure transducers 140A-140D may be configured to detect an inhalation pressure of each inhalation stream provided to the patients 1-4. The differential pressure transducers 142A-142D may be configured to detect an inhalation/exhalation pressure differential (e.g., the pressure difference between the inhalation pressures in the respective inhalation lines 212A-212D and exhalation pressures in the corresponding exhalation lines 222A-222D). In other words, the differential pressure transducers 142A-142D may be configured to detect a pressure differential between the inhalation pressure and an exhalation pressure for each of the patients 1-4, with the exhalation pressure being the pressure of an exhalation stream exhaled from each of the patients 1-4 into the respective exhalation lines 222A-222D.

Referring to FIG. 1D, the VSMS 300 may include a power receptacle 150, an AC/DC inverter 152, a sense card 154, an analog/digital converter (ADC) 156, and a central processing unit (CPU) 158 (e.g., computer or logic circuit). The power receptacle 150 may include a ground line (G) connected to ground (GND1), a fuse F1 (e.g., a 500 mA fuse) on the live power line (L) and a dual pole single throw switch on both the live and neutral lines (L, N) to control the flow of power into the AC/DC inverter 152. The power receptacle 150 may connect the AC/DC inverter 152 to an external AC power source. The AC/DC inverter 152 may comprise a 5V, 30 W inverter which converts AC power to DC power and provides the DC power to the sense card 154.

The ADC 156, the gauge pressure transducers 140A-140D, and the differential pressure transducers 142A-142D, may be disposed on the sense card 154 and electrically connected to each other and to the CPU 158. The ADC 156 may receive analog signals from the gauge pressure transducers 140A-140D and 142A-142D, and output corresponding digital signals to the CPU 158. The CPU 158 may calculate various ventilation parameters for each patient, such as each patient's pressure profile, flow rate, and volume exchange. The CPU 158 may output corresponding video signals to a monitor 160, in order to display detected ventilation parameters for each patient. An operator may then adjust the pressure control valve 120A-120D of a corresponding patient, without changing the flow settings for other patients, based on the displayed ventilation parameters. An optional fuse F2 (e.g., a 1 Amp fuse) may be located on the positive power bus on the sense card 154.

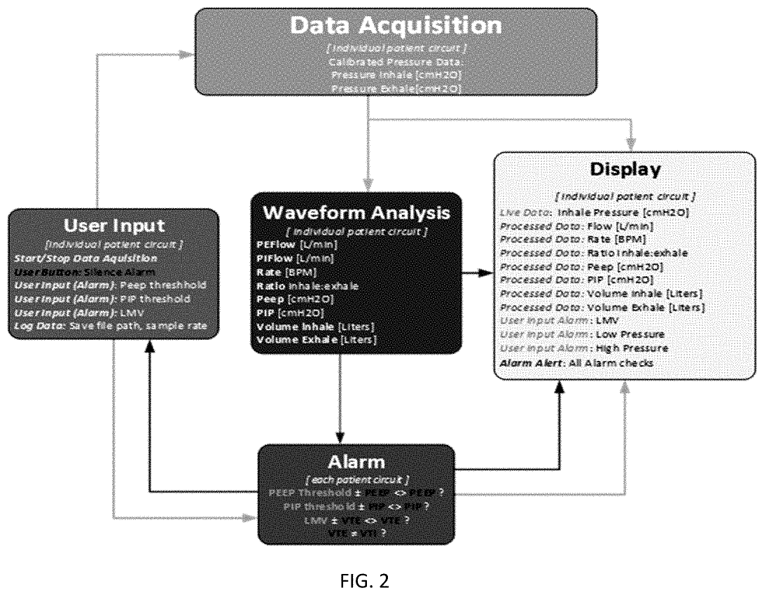

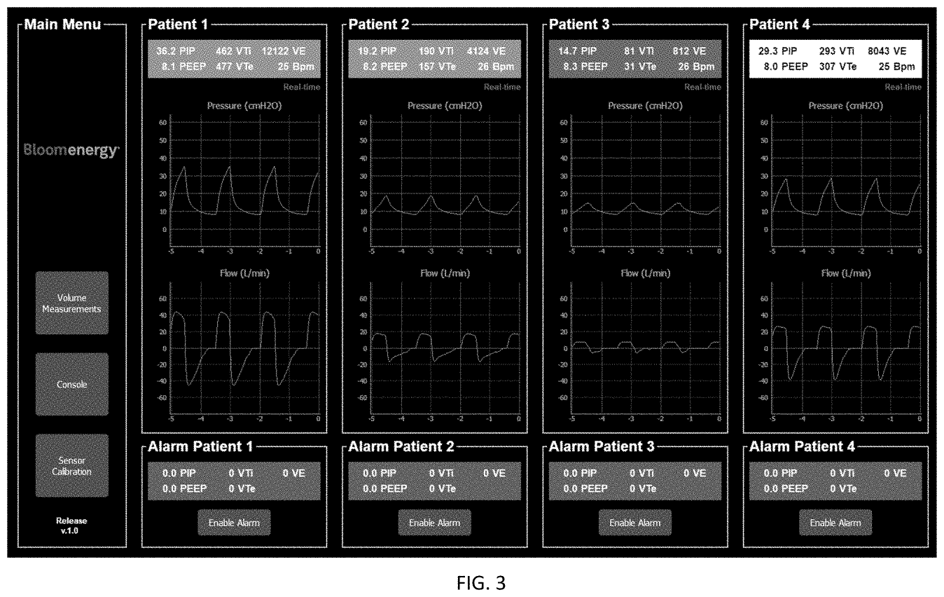

FIG. 2 shows VSMS data flow from user input, data acquisition to estimate and display, and FIG. 3 is a screen shot showing ventilation parameters that may be output by the VSMS 300, according to various embodiments of the present disclosure.

Referring to FIG. 2, data such as calibration data, inhalation pressure, exhalation pressure, and pressure differential can be acquired by the VSMS from individual patients. Data may also be input by a user. For example, a user may set alarm thresholds and patient data. Waveform data analysis can be performed and the results of the analysis may be displayed. For example, as shown in FIG. 3, critical ventilation parameters including breaths per minute (BPM), peak inspiratory pressure (PIP), positive end-expiratory pressure (PEEP), tidal volume inhale (VTI), tidal volume exhale (VTE), volume exhale (VE), combinations thereof, or the like, may be displayed.

According to various embodiments, a single medical ventilator 50 controls the respiratory rates of all patients connected to the ventilator through the splitter module 100. The patients are intubated, heavily sedated and paralyzed. The splitter module provides independent control of PIP, PEEP, and tidal volume for each patient using a pressure control valve 120A-120D for each patient, without significantly affecting the ventilation parameters of other patients connected to the same ventilator. Patients can be added or removed at any time without significantly affecting the ventilation parameters of other patients. Herein, “significantly affecting ventilation parameters” may refer to changing one or more ventilation parameters by more than 5%, such as by more than 3%, or more than 1%.

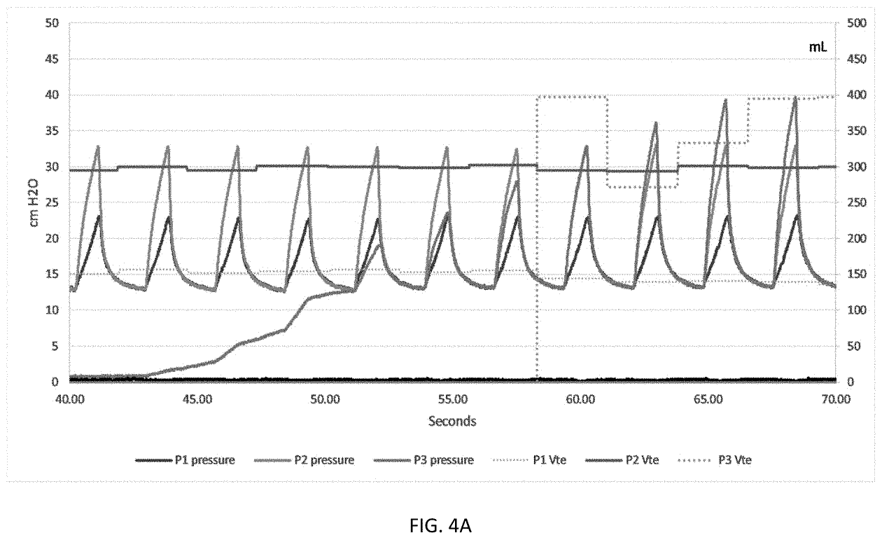

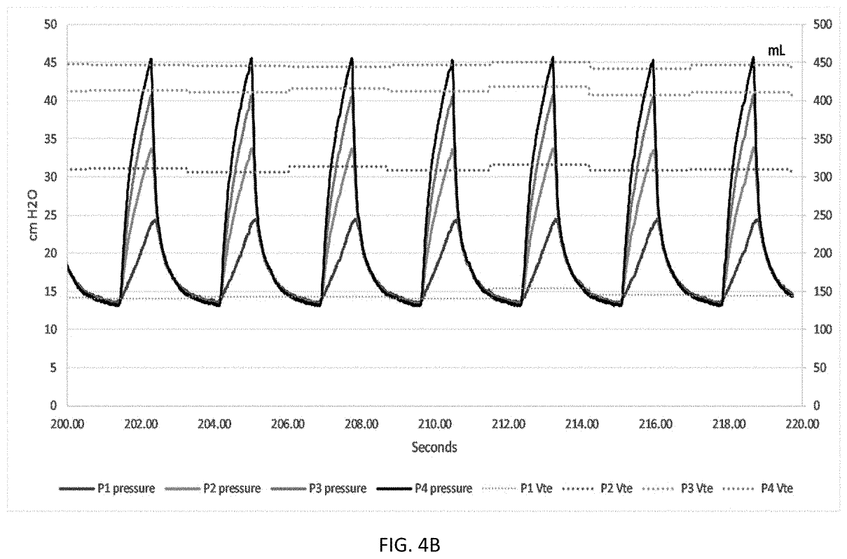

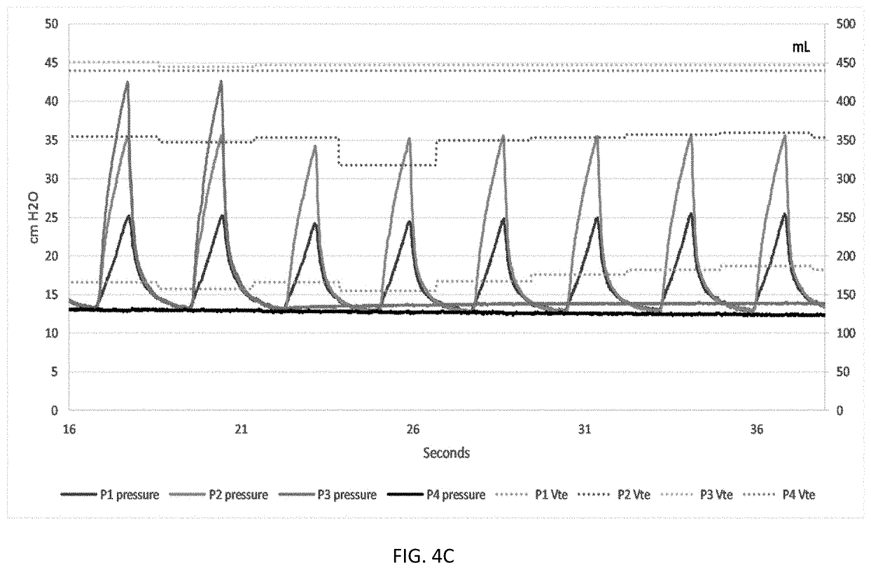

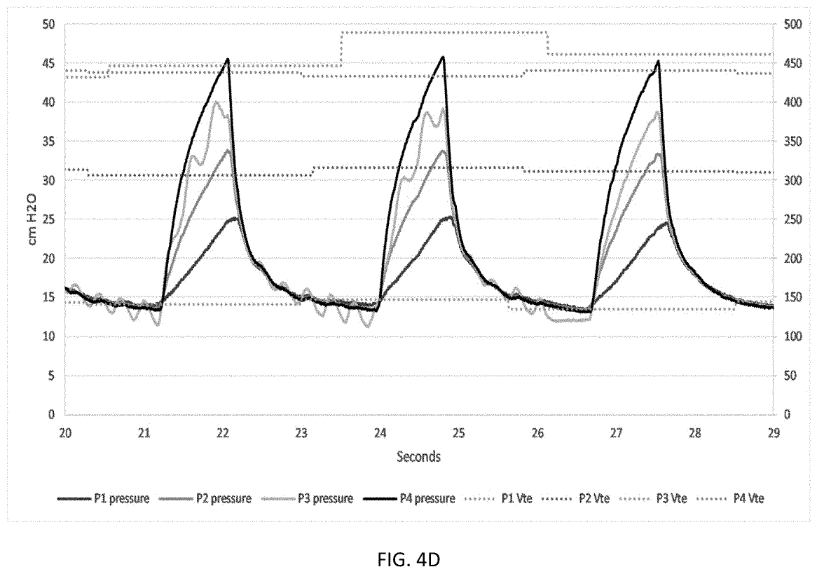

In one embodiment, the splitter device provides in-line monitoring of PIP, PEEP, and tidal volume for all patients connected to the same ventilator through the splitter device to assist easy patient-specific adjustments. FIGS. 4A-4D are graphs that may be output by the VSMS during in-line monitoring of patients.

Referring to FIG. 4A, the addition of a third patient (P3) to the VSMS has little impact on the ventilation pressure and VTE of first and second patients (P1, P2) already connected to the VSMS. Referring to FIG. 4B, the VSMS may provide stable ventilation pressure and VTE for four connected patients (P1-P4).

Referring to FIG. 4C, disconnecting a third patient (P3) from the VSMS 300 may have little impact on the ventilation pressure and VTE of first and second patients (P1, P2) connected to the VSMS. Referring to FIG. 4D, an intentional pressure disturbance applied to a third patient P3 may have little impact on the ventilation pressure and VTE of the remaining patients connected to the VSMS.

Alarms may be triggered under suboptimal conditions. The central processing unit (e.g., computer) 158 with external display 160 may show statistics for individual patients. Thresholds can be set by the operator for ±PEEP, Positive End-Expiratory Pressure (cmH₂O), ±PIP, Peak Inspiratory Pressure (cmH₂O), and ±VTA % [VTI/VTE*100] (L), for each patient. If computed values fall below or above thresholds, an audible alarm may sound, and/or the display 160 may display a red signal to show which patient circuit needs attention. Alarms can be silenced or reset. If the alarm is silenced, a watchdog timer may re-enable the silenced alarm after 2 minutes. If an alarm is reset, and values are outside of the threshold range on the next breathing cycle, the alarm may activate again.

Preferably, the plural patients should be on similar settings with regard to respiratory rate and FiO₂ as this is shared amongst all patients on the splitter module 100. While inspiratory pressure, tidal volume, and PEEP can be adjusted to each patient specifically, to the extent possible, selecting patients who have more similar ventilator settings is preferred.

Preferably, patients should be initiated on mechanical ventilation on a separate ventilator until they are on stable ventilator settings. This allows clinicians to obtain their ideal inspiratory pressures, tidal volume, and lung compliance. These numbers allow safer introduction and titration into the splitter module 100.

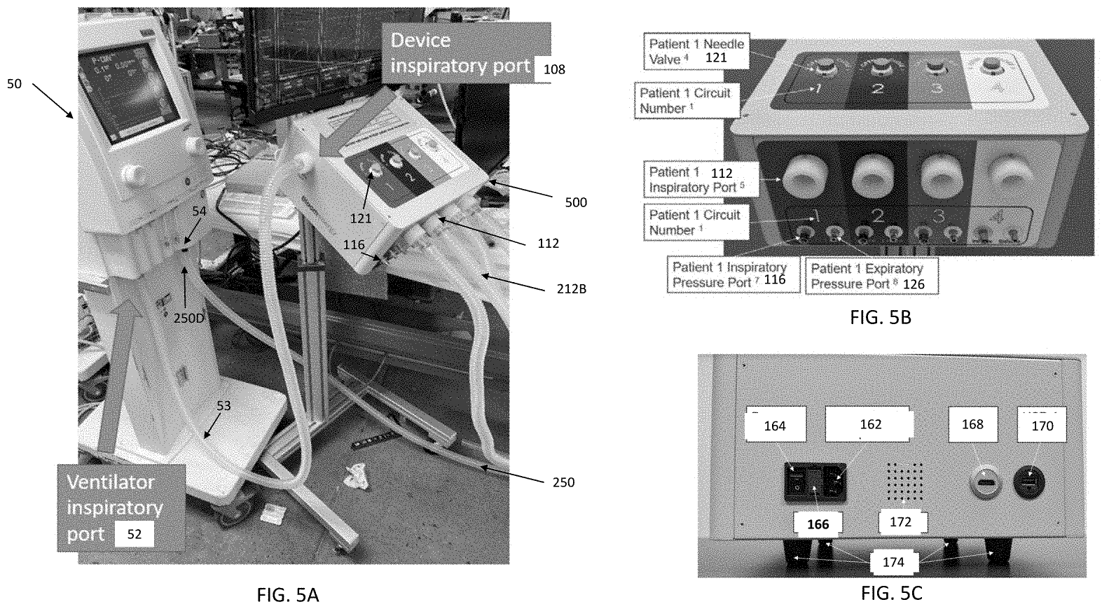

FIGS. 5A-5C are photographs showing outer surfaces of a splitter module 500, according to various embodiments of the present disclosure, that exemplifies the splitter module 100 of FIGS. 1A-1C. Referring to FIGS. 1B, 1C and 5A-5C, the splitter module 500 may include a module inspiratory port 108, patient inhalation (i.e., inspiratory) ports 112, valve controls 121 which control the valves 120A-120D, inhalation pressure ports 116, and exhalation pressure ports 126 for the respective transducers 140 and 142. The module inspiratory port 108 may be configured to connect the splitter module 500 to a ventilator line 53 which connects to the ventilator 50 inhalation port 52 to the splitter module 500. The module ventilator port 108 may be fluidly connected to the inhalation manifold 200. The splitter module 500 may also include a power cord receptacle 162, a power switch 164, a fuse 166, an HDMI port 168, a USB port 170, a vent 172, and supports (e.g., feet) 174.

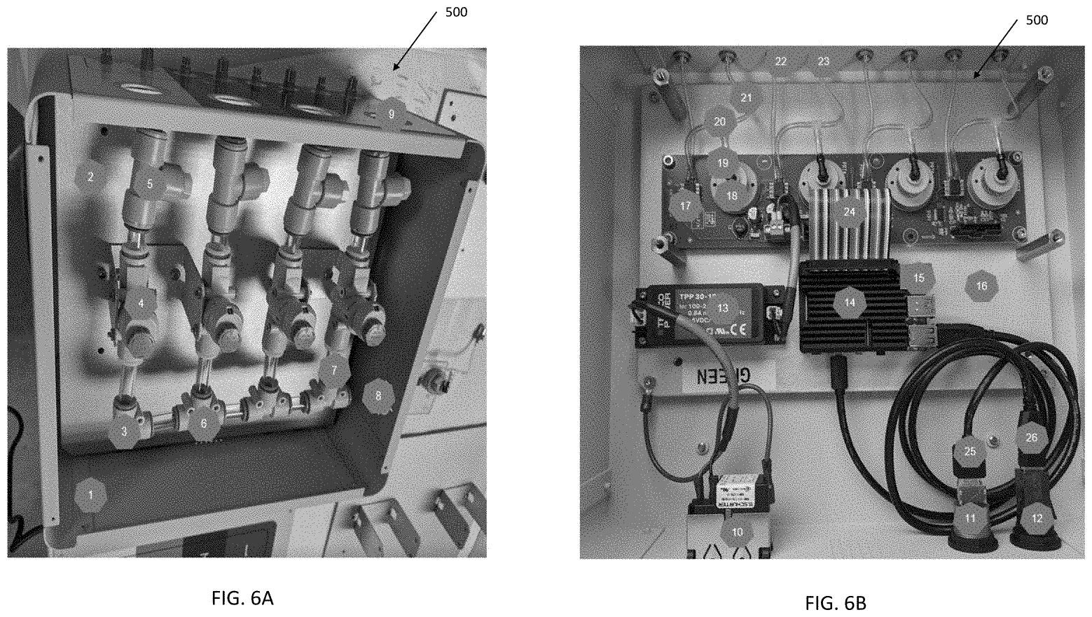

FIG. 6A is a photograph showing the exemplary splitter module 500 with its cover removed, and 6B is a photograph showing the exemplary splitter module 500 with its cover and mid plane removed, according to various embodiments of the present disclosure. A parts list of the exemplary splitter module 500 is provided in Table 1 below.

TABLE 1

| Item | | | |
|---|---|---|---|
| # | Description | Qty | Material |
| 1 | Enclosure | 1 | Aluminum |
| 2 | Mid Plane | 1 | Aluminum |
| 3 | Elbow ½″ push-to-connect | 1 | PBT, SS |
| 4 | Needle Valve ½″ push-to- | 4 | PBT, SS |
| | connect | | |
| 5 | Relief valve ½″ push-to- | 4 | PBT, SS |
| | connect | | |
| 6 | Tee ½″ push-to-connect | 3 | PBT, SS |
| 7 | ½″ tubing | 3 ft | PVC |
| 8 | ½″ to 22 mm ID fitting | 4 | Polypropylene |
| 9 | ½″ to 22 mm ID fitting | 1 | Polypropylene |
| 10 | AC Power Receptacle | 1 | |
| 11 | HDMI pass thru connector | 1 | |
| 12 | USB-A pass thru connector | 1 | |
| 13 | Power Supply | 1 | |
| 14 | Heatsink Case for Raspberry | 1 | Aluminum |
| | Pi 4 B | | |
| 15 | Raspberry Pi 4B | 1 | |
| 16 | Backplane | 1 | Aluminum |
| 17 | Differential transducer | 1 | |
| 18 | Pressure Transducer | 4 | — |
| 19 | ⅛″ tubing | 1 ft | PVC |
| 20 | Tee, Barb ⅛″ to 1/16″ | 4 | Nylon |
| 21 | 1/16″ tubing | 3 ft | PVC |
| 22 | ⅛″ female luer panel mount | 4 | SS |
| 23 | ⅛″ male luer panel mount | 4 | SS |
| 24 | Cable, flat ribbon 40 pin | 1 | — |
| | GPIO | | |
| 25 | Cable, Micro HDMI to HDMI | 1 | — |
| 26 | Cable, USB-A Male to USB- | 1 | — |
| | A Male | | |

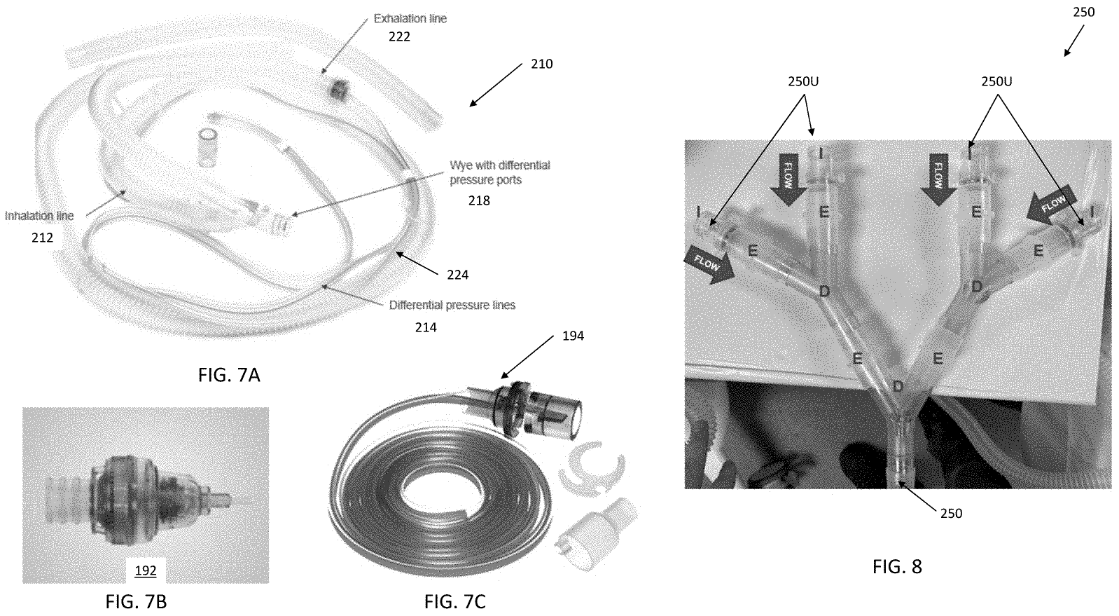

As shown in FIGS. 6A and 6B, the splitter module 500 may include plural pressure relief valves 5 (in place of the common relief outlet 202 in FIG. 1C), T-shaped junctions 6 and elbow junction 3 (which correspond to the junctions 213A-213D in FIGS. 1A and 1C), and tubing 7 that form the inhalation manifold 200 of FIGS. 1A-1C that splits incoming air from the ventilator into separate streams. The tubing 19, barb tees 20, and tubing 21, in combination with the differential transducers 17 and pressure transducers 18 read (i.e., detect) differential pressure across a wye connector 218 of a ventilation circuit 210 (see FIG. 7A). The detected differential pressure is converted by a processor 15 (e.g., computer or logic circuit, such as the CPU 158) to a numerical value which is used to display each patient's pressure profile, flow rate, and volume exchange, as shown in FIG. 3. The operator may then adjust the corresponding needle valve 4 (i.e., 120) setting for a specific patient, based on the displayed ventilation data without changing the valve setting for the needle valves 4 of other patients connected to the splitter module 500.

FIG. 7A is a photograph of a patient ventilation circuit 210, FIG. 7B is a photograph of a one-way valve, and FIG. 7C is a photograph of a pressure line, according to various embodiments of the present disclosure.

Referring to FIGS. 5A-5C and 7A-7C, the patient ventilation circuit 210 may include an inhalation line 212 (e.g., one of inhalation lines 212A-212D), an exhalation line 222 (e.g., one of exhalation lines 222A-222D), a wye connector 218 (i.e., a three-way connector or three-branched device, shaped like the letter “y”) including differential pressure ports, an inhalation pressure line 214 (e.g., one of inhalation pressure lines 214A-214D), and exhalation pressure line 224 (e.g., one of exhalation pressure lines 224A-224D). The patient ventilation circuit 210 may also include a variable exhalation PEEP valve 192 (17921-001) and a Hamilton proximal flow sensors 194 (PN 282051) fluidly connected thereto.

A first end of the inhalation line 212 may be connected to one of the inhalation ports 112, and a second end of the inhalation line 212 may be connected to the wye connector 218. A first end of the exhalation line 222 may be connected to the wye connector 218, and a second end of the exhalation line 222 the other may be connected to an exhalation manifold 250, as discussed below with respect to FIG. 8.

A first end of the inhalation pressure line 214 may be connected to a differential pressure port of the wye connector 218, and a second end of the inhalation pressure line 214 may be connected to one of the inhalation pressure ports 116. A first end of the exhalation pressure line 224 may be connected to a differential pressure port of the wye connector 218, and a second end of the exhalation pressure line 224 may be connected to one of the exhalation pressure ports 126.

FIG. 8 is a photograph of an exhalation manifold 250, according to various embodiments of the present disclosure. Referring to FIG. 8, the exhalation manifold 250 may include multiple upstream ends 250U and a single downstream end 250D. Each upstream end 250U may be connected to a respective exhalation line 222 (e.g., 222A-222D), and the downstream end 250D may be fluidly connected to the exhalation port 54 of a ventilator 50. Accordingly, the exhalation manifold may collect air exhaled from multiple patients, such that the exhaled air may be provided to the same exhalation port 54.

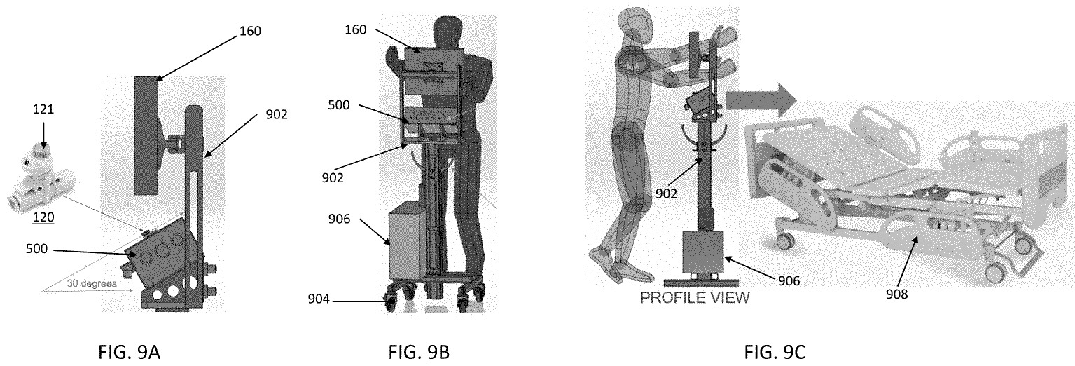

FIGS. 9A, 9B, and 9C are side and perspective views of a mounted splitter module 500, according to various embodiments. As shown in FIGS. 9A-9C the splitter module 500 may be mounted on a movable pedestal stand 902 having wheels 904. One or more of a display monitor 160, a computer 906, a control panel (which may be in the display monitor), and/or a keyboard may be mounted on the stand 902 to occupy a minimal footprint.

Needle valve 120 controls 121 and the dial indicator values are presented to the ventilator operator at an ergonomic 30 degree angle. The needle valve 120 settings can be preset to match a patient's lung compliance prior to introducing that patient to the breathing circuit (i.e., intubating the patient) and adjusted periodically for each patient during the course of the therapy session. The patient inhalation lines (depicted by the large arrow in FIG. 9C) may be presented at hospital bed 908 height.

According to various embodiments, a splitter module 100, 500 minimizes interactions between patients connected to the same ventilator 50, such patients can be added, removed, and/or have ventilation parameters adjusted, without impacting the remaining patients connected to the same ventilator. Unique control parameters and alarms may be set for each patient.

According to various embodiments, the splitter module 500 may be located in a housing 1 having an integrated design which is configured to minimize dead space. In this embodiment, the needle valves 120 are separated from the pressure transducers 140, 142. The components are packaged in such a way that the patient inhalation lines and pressure transducer lines come out in the same direction from the splitter module and can be bundled neatly.

In one embodiment, the splitter module provides unidirectional airflow and adequate dead space to avoid cross-contamination. The splitter module may be used with all models of ventilators used in hospitals, including open and closed loop systems. In one embodiment, a single ventilator connected to the splitter module (e.g., by a tube or conduit) is operated using “pressure limited” or “pressure control” protocol and set at highest pressure needed to support all patients.

While the splitter module 100, 500 and the ventilator 50 may be located in separate housings in some embodiments, in other embodiments the splitter module and ventilator may be located in the same housing (e.g., same box).

The preceding description of the disclosed aspects is provided to enable any person skilled in the art to make or use the present invention. Various modifications to these aspects will be readily apparent to those skilled in the art, and the generic principles defined herein may be applied to other aspects without departing from the scope of the invention. Thus, the present invention is not intended to be limited to the aspects shown herein but is to be accorded the widest scope consistent with the principles and novel features disclosed herein.

What is claimed is:

1. A splitter module configured to connect a single medical ventilator to multiple intubated patients,
wherein the splitter module is configured to independently control at least one ventilation parameter for each of the patients, such that modifying a ventilation parameter of one of the patients does not significantly affect the ventilation parameters of the other patients.

2. The splitter module of claim 1, wherein the at least one ventilation parameter comprises breaths per minute, peak inspiratory pressure, positive end-expiratory pressure, inhale tidal volume, exhale tidal volume, exhale volume, or a combination thereof.

3. The splitter module of claim 1, wherein the splitter module is configured to modify a ventilation parameter of one of the patients without significantly affecting the ventilation parameters of the other patients.

4. The splitter module of claim 1, further comprising:
an inhalation manifold configured to divide an inspiratory air stream received from the single medical ventilator into separate inhalation streams that are respectively provided to the patients; and
pressure control valves configured to respectively control a flow rate of each of the inhalation streams.

5. The splitter module of claim 4, wherein the pressure control valves comprise needle valves.

6. The splitter module of claim 4, further comprising:
gauge pressure transducers configured to respectively detect inhalation pressures of the inhalation streams; and
differential pressure transducers configured to respectively detect pressure differentials between the inhalation pressures and exhalation pressures of respective exhalation streams exhaled from the patients.

7. A ventilator sharing and monitoring system (VSMS), comprising:
the splitter module of claim 4;
the single medical ventilator fluidly connected to the splitter module;
plural inhalation lines fluidly connected to the splitter module and configured to provide the inhalation streams to respective intubated patients;
plural exhalation lines configured to receive respective exhalation streams exhaled from the patients; and
an exhalation manifold fluidly connected to the plural exhalation lines and to the medical ventilator, and configured to combine the exhalation streams into an expiration stream, and provide the expiration stream to the single medical ventilator.

8. A ventilator sharing and monitoring system (VSMS), comprising:
a medical ventilator;
a splitter module fluidly connected to the medical ventilator, the splitter module comprising an inhalation manifold configured to divide an inspiration stream received from the medical ventilator into separate inhalation streams;
plural inhalation lines fluidly connected to the splitter module and configured to provide the inhalation streams to respective intubated patients;
plural exhalation lines configured to receive respective exhalation streams exhaled from the patients; and
an exhalation manifold fluidly connected to the plural exhalation lines and to the medical ventilator, and configured to combine the exhalation streams into an expiration stream, and provide the expiration stream to the medical ventilator.

9. The VSMS of claim 8, further comprising:
one-way inhalation valves fluidly connected to the plural inhalation lines and configured to prevent air from returning to the inhalation manifold from the inhalation lines;
one-way exhalation valves fluidly connected to the plural exhalation lines and configured to prevent air from returning to the exhalation lines from the exhalation manifold; and
filters connected to the plural inhalation lines and configured to filter the inhalation air streams prior to the inhalation air streams being provided to the patients.

10. The VSMS of claim 8, further comprising:
wye connectors fluidly connecting the patients to a respective one of the plural inhalation lines and a respective one of the plural exhalation lines;
inhalation pressure lines respectively fluidly connecting the wye connectors to the ventilator splitter module; and
exhalation pressure lines respectively fluidly connecting the wye connectors to the ventilator splitter module.

11. The VSMS of claim 10, wherein the splitter module further comprises:
gauge transducers respectively fluidly connected to the plural inhalation pressure lines and configured to detect an inhalation pressure for each patient; and
differential transducers fluidly connected to the plural exhalation pressure lines and the plural inhalation pressure lines, the differential pressure transducers configured to detect a pressure differential between the inhalation pressure and an exhalation pressure of each of the patients.

12. The VSMS of claim 11, wherein the splitter module further comprises a central processing unit (CPU) configured to receive pressure data from the gauge transducers and the differential transducers, and calculate ventilation parameters for each patient.

13. The VSMS of claim 12, wherein:
the ventilation parameters comprise comprises breaths per minute, peak inspiratory pressure, positive end-expiratory pressure, inhale tidal volume, exhale tidal volume, exhale volume, or a combination thereof; and
the CPU is configured to perform a waveform analysis to generate the ventilation parameters.

14. The VSMS of claim 13, further comprising a monitor and a user input unit, wherein the CPU is configured to output the ventilation parameters to the monitor and set alarms based on user input received through the user input unit.

15. The VSMS of claim 8, wherein the medical ventilator and the splitter module are disposed in a same housing or in different housings.

16. A medical ventilation method, comprising:
connecting a single medical respirator to intubated patients through a splitter module; and
adjusting a ventilation parameter of one of the patients using the splitter module, without significantly affecting ventilation parameters of the remaining patients.

17. The method of claim 16, wherein the splitter comprises:
pressure transducers configured to detect inhalation pressures and inhalation/exhalation pressure differentials; and
pressure control valves configured to respectively control inhalation flow rates for the patients.

18. The method of 17, further comprising:
detecting pressure differentials across wye connectors fluidly connected to each patient using the respective pressure transducers;
displaying at least one ventilation parameter for each of the patients; and
changing a setting of the pressure control valve for one of the patient, based on the at least one displayed ventilation parameter for that patient, without significantly affecting the ventilation parameters of the other patients.

19. The method of claim 16, further comprising disconnecting one of the patients from the splitter module, without substantially affecting the ventilation parameters of the remaining patients.

20. The method of claim 16, further comprising connecting an additional patient to the splitter module, without substantially affecting the ventilation parameters of the remaining patients.
